(19) United States
(12) Patent Application Publication
(10) Pub. No.: US 2025/0235423 A1
(43) Pub. Date: Jul. 24, 2025

(54) USES OF AMINO ACID-CHELATED ZINC, INCLUDING IMMUNE REGULATION AND TREATMENT OF AUTOIMMUNE DISEASE

(71) Applicant: FAAC Bio Inc., New Taipei City (TW)

(72) Inventors: Hsin-Ching HSU, New Taipei City (TW); Li-Chuan HSU, New Taipei City (TW); Wei-Yu LIN, New Taipei City (TW); Hao-Chuan FU, New Taipei City (TW)

(21) Appl. No.: 19/022,312

(22) Filed: Jan. 15, 2025

Related U.S. Application Data
(60) Provisional application No. 63/622,205, filed on Jan. 18, 2024.

(30) Foreign Application Priority Data
Nov. 18, 2024 (TW) 113144326

(51) Int. Cl. A61K 31/315; A61P 37/06; C12N 5/0784

(52) U.S. Cl. CPC A61K 31/315; A61P 37/06; C12N 5/0639

(57) ABSTRACT

A method for regulating immunity and/or inhibiting dendritic cell maturation is provided, especially for treating an allergic disease, treating an autoimmune disease, and/or preventing an organ transplant rejection. Also provided is a combination for preparing tolerogenic dendritic cells, comprising (1) an amino acid-chelated zinc, and (2) a dendritic cell medium.

### CLAIM FOR PRIORITY

This application claims the benefits of U.S. Patent Provisional Application No. 63/622,205 filed on Jan. 18, 2024 and Taiwan Patent Application No. 113144326 filed on Nov. 18, 2024, the subject matters of which are incorporated herein in their entirety by reference.

### FIELD OF THE INVENTION

The present invention relates to use of amino acid-chelated zinc, particularly the use of an amino acid-chelated zinc on immune regulation in a subject. The present invention especially relates to providing a subject in need, such as patients with autoimmune disease and organ transplant recipients, with the benefit(s) of inhibiting immune response.

### BACKGROUND OF THE INVENTION

According to statistics made by the Autoimmune Association in the USA, more than 100 autoimmune diseases have been discovered and more than fifty million people in the USA are suffering from autoimmune diseases. Moreover, the number of patients suffering from organ failure is increasing year by year due to the gradual aging of social structure and changes in modern lifestyle. Clinically, organ transplant is the most effective method for treating an end-stage organ failure. It is expected that the global organ transplantation market will reach US$61.5 billion by 2027 and increase at a compound annual growth rate of 9.9% over the forecast period. To prevent the recipient's immune system from rejecting the transplanted organ, the recipient usually needs to take an immunosuppressant over a long period of time. Therefore, the industry is committed to developing medicaments that are effective in regulating immunity, particularly inhibiting undesired immune response(s).

Dendritic cell (DC) is a professional antigen-presenting cell (APC), which serves as a bridge between the innate immune response and the adaptive immune response. When an exogenous antigen is recognized by the immune system, DC will be activated and the expressions of costimulatory factors (e.g., CD80, CD86 and CD40) and major histocompatibility complex class II (MHC II) on the surface of DC will be increased. Furthermore, the activated DC will also secret proinflammatory cytokines to activate downstream T cells.

In recent years, it was found from studies that DC with low expressions of costimulatory factors and MHC II (i.e., semi-mature DC) is able to induce immune tolerance, thereby allowing T cell anergy, activating and regulating regulatory T cells (Treg cells), and secreting anti-inflammatory factors. Such semi-mature DC is also known as tolerogenic DC. Because of the anti-inflammatory and immunosuppressive effects of tolerogenic DC, tolerogenic DC is also considered as a potential cell therapy for treating allergic diseases, treating autoimmune diseases (such as type 1 diabetes, multiple sclerosis, rheumatoid arthritis, etc.) or preventing an organ transplant rejection.

### SUMMARY OF THE INVENTION

Inventors of the present invention found that amino-chelated zinc has an excellent effect on inhibiting dendritic cell maturation. As compared to the known active ingredients capable of inhibiting dendritic cell maturation (e.g., Zinc sulfate), the amino-chelated zinc can provide a desired inhibitory effect at a relatively low concentration of zinc. Inventors of the present invention also found that the administration of amino acid-chelated zinc in a subject with an autoimmune disease can effectively alleviate the subject's symptom(s) resulted from the autoimmune disease.

Therefore, an objective of the present invention is to provide the use of an amino acid-chelated zinc in the manufacture of a composition, wherein the composition is used for immune regulation. In some embodiments, the composition is a pharmaceutical composition or a food composition. In some embodiments, the pharmaceutical composition is for treating an allergic disease, treating an autoimmune disease, and/or preventing an organ transplant rejection.

In some embodiments of the use in accordance with the present invention, the composition is an aqueous composition with a pH less than 7.

Another objective of the present invention is to provide the use of an amino-chelated zinc in the manufacture of a composition, wherein the composition is for inhibiting dendritic cell maturation.

Still another objective of the present invention is to provide a combination, comprising:
(1) an amino acid-chelated zinc; and
(2) a dendritic cell medium.

In some embodiments of the combination in accordance with the present invention, the combination further comprises a dendritic cell.

In some embodiments of the combination in accordance with the present invention, the amino acid-chelated zinc has an amount of zinc ranging from 1 mg to 25 mg per mL of the dendritic cell medium.

Still another objective of the present invention is to provide a method for regulating immunity and/or inhibiting dendritic cell maturation, which comprises administering an effective amount of the composition as mentioned above to a subject in need.

In some embodiments of the method for immune regulation in accordance with the present invention, the method is for treating an allergic disease, treating an autoimmune disease, and/or preventing an organ transplant rejection.

Still another objective of the present invention is to provide a method for preparing a tolerogenic dendritic cell, comprising contacting the tolerogenic dendritic cell with an effective amount of the amino acid-chelated zinc. In some embodiments of the method, the contact is performed by culturing the dendritic cell in a dendritic cell medium containing the amino acid-chelated zinc.

In some embodiments of the use, combination and method in accordance with the present invention, the amino acid-chelated zinc has a molar ratio of zinc and amino acid ranging from 1:1 to 1:10.

In some embodiments of the use, combination and method in accordance with the present invention, the amino acid-chelated zinc is selected from the group consisting of glycine-chelated zinc, alanine-chelated zinc, valine-chelated zinc, leucine-chelated zinc, isoleucine-chelated zinc and combinations thereof.

To render the above objectives, technical features and advantages of the present invention more apparent, the present invention will be described in detail with reference to some embodiments hereinafter.

### BRIEF DESCRIPTION OF THE DRAWINGS

FIG. 1 is a graphical representation of flow cytometry results for analyzing the expression level of CD11c in the isolated mouse bone marrow dendritic cells (BMDCs), wherein 97.22% of the cells expressed CD11c and only 2.78% of the cells didn't express CD11c. This result indicates that the purity of BMDCs is relatively high.

FIGS. 2A to 2K are graphical representations of flow cytometry results for analyzing the expression levels of CD80, CD83, CD86, CD40, CD11c and MHC II on the cell surfaces of BMDCs with different treatments, wherein FIG. 2A shows the result of untreated BMDCs (i.e., immature DC), FIG. 2B shows the result of activated BMDCs (i.e., mature DC), FIG. 2C shows the result of BMDC treated with zinc sulfate at a zinc concentration of 3.27 mg/mL, FIG. 2D shows the result of BMDC treated with zinc sulfate at a zinc concentration of 6.54 mg/mL, FIG. 2E shows the result of BMDC treated with zinc sulfate at a zinc concentration of 13.08 mg/mL, FIG. 2F shows the result of BMDC treated with a solution of glycine-chelated zinc (CMC-Zn) at a zinc concentration of 3.27 mg/mL, FIG. 2G shows the result of BMDC treated with CMC-Zn at a zinc concentration of 6.54 mg/mL, FIG. 2H shows the result of BMDC treated with CMC-Zn at a zinc concentration of 13.08 mg/mL, FIG. 2I shows the result of BMDC treated with modified CMC-Zn at a zinc concentration of 3.27 mg/mL, FIG. 2J shows the result of BMDC treated with modified CMC-Zn at a zinc concentration of 6.54 mg/mL and FIG. 2K shows the result of BMDC treated with modified CMC-Zn at a zinc concentration of 13.08 mg/mL.

FIG. 3 is a line chart depicting the average weight percentage of both paws of rats as a function of time, wherein the control group includes the untreated healthy rats, the CIA group includes rats with collagen-induced arthritis, the MTX group includes arthritis rats administered with MTX from Day 17, the CMC-Zn group includes arthritis rats administered with the CMC-Zn from Day 17, and the modified CMC-Zn group includes arthritis rats administered with the modified CMC-Zn from Day 17.

FIG. 4 is a bar chart showing the average weight percentage of both paws of rats on Day 24, wherein the rats in each group were treated in the same way as those of FIG. 3. “*” represents that the result has a significant difference to that of the control group (p value<0.05), and “#” represents that the result has a significant difference to that of the CIA group (p value<0.05).

### DESCRIPTION OF THE PREFERRED EMBODIMENT

Hereinafter, some embodiments of the present invention will be described in detail. However, the present invention may be embodied in various embodiments and should not be limited to the embodiments described in the specification.

Unless it is additionally explained, the expressions “a,” “the,” or the like recited in the specification (especially in the claims) should include both the singular and the plural forms; the term “subject” recited herein refers to a human or a non-human mammal. Examples of the non-human mammal include, but are not limited to, bovine, horse, sheep, goat, pig, donkey, mule, dog, cat, rabbit, rodent (such as hamster, guinea pig), ape, monkey, and orangutan.

The numerical ranges (e.g., 5 to 100) used herein should be construed as including all of the rational numbers within the ranges and ranges consisting of any rational numbers within the ranges. Therefore, the numerical ranges used herein should include all the possible combinations of numerical values between the listed minimum and maximum values.

As mentioned above, inventors of the present invention found that the amino-chelated zinc has excellent effect on inhibiting dendritic cell maturation, and the administration of amino-chelated zinc to a subject with an autoimmune disease can alleviate the subject's symptom(s) resulted from the autoimmune disease. Therefore, the present invention relates to the use of the amino acid-chelated zinc on regulating immunity and/or inhibiting dendritic cell maturation.

### Amino Acid-Chelated Zinc

The amino acid-chelated zinc suitable for the present invention can be a commercially available product or a product of a synthesis method. Methods suitable for synthesizing the amino acid-chelated zinc are well known in the art. For example, the chelation of amino acid and zinc can be performed by operations such as physically mixing and/or heating to provide the amino acid-chelated zinc. The molar ratio of zinc to amino acid in the amino acid-chelated zinc in accordance with the present invention may range from 1:1 to 1:10, such as 1:1, 1:2, 1:3, 1:4, 1:5, 1:6, 1:7, 1:8, 1:9 or 1:10. In some embodiments, the amino acid-chelated zinc has a molar ratio of zinc to amino acid ranging from 1:1 to 1:4.

There is no specific limitation on the amino acid used in the preparation of the amino acid-chelated zinc for the present invention as long as the desired effect(s) of the present invention (i.e. regulating immunity and/or inhibiting dendritic cell maturation) is not affected. Examples of suitable amino acid for the preparation of the amino acid-chelated zinc include, but are not limited to, glycine, alanine, valine, leucine, isoleucine and combinations thereof. Alternatively, the amino acid-chelated zinc used in the present invention can be prepared from a zinc-containing inorganic compound. Examples of the zinc-containing inorganic compound include, but are not limited to, zinc sulfate, zinc carbonate, zinc oxide, zinc chloride and combinations thereof.

In some embodiments, the amino acid-chelated zinc used in the present invention can be prepared by sequentially conducting the steps of mixing an amino acid and a zinc source, refrigerating and heating. Preferably, the refrigerating step comprises placing the mixture obtained from the mixing step at 0° C. to 4° C. for at least 8 hours, such as placing at 0° C. to 4° C. for 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 36 hours, or 48 hours, or within a range between any two of the values described herein. Preferably, the heating step comprises heating the refrigerated mixture at 50° C. to 100° C. for 6 hours to 24 hours, such as heating the refrigerated mixture at 50° C., 52.5° C., 55° C., 57.5° C., 60° C., 62.5° C., 65° C., 67.5° C., 70° C., 72.5° C., 75° C., 77.5° C., 80° C., 82.5° C., 85° C., 87.5° C., 90° C., 92.5° C., 95° C., 97.5° C. or 100° C. for 6 hours, 6.5 hours, 7 hours, 7.5 hours, 8 hours, 8.5 hours, 9 hours, 9.5 hours, 10 hours, 10.5 hours, 11 hours, 11.5 hours, 12 hours, 12.5 hours, 13 hours, 13.5 hours, 14 hours, 14.5 hours, 15 hours, 15.5 hours, 16 hours, 16.5 hours, 17 hours, 17.5 hours, 18 hours, 18.5 hours, 19 hours, 19.5 hours, hours, 20.5 hours, 21 hours, 21.5 hours, 22 hours, 22.5 hours, 23 hours, 23.5 hours or 24 hours. Alternatively, the heating temperature and heating time can be within a range between any two of the values described herein. In some embodiments, a drying step (e.g., thermal drying or lyophilization) can be further conducted after the heating step to provide the amino acid-chelated zinc in powder form.

### Composition for Regulating Immunity/Inhibiting Dendritic Cell Maturation

As mentioned above, the amino-chelated zinc is effective in regulating immunity and/or inhibiting dendritic cell maturation and thus can be used to provide a composition for regulating immunity and/or inhibiting dendritic cell maturation.

Therefore, in an aspect of the present invention, a composition for regulating immunity and/or inhibiting dendritic cell maturation is provided, wherein the composition comprises an effective amount of the amino acid-chelated zinc. In some embodiments of the composition provided in accordance with the present invention, the amino acid-chelated zinc can be mixed with water (e.g., sterile water, pure water) or an aqueous solution to form an aqueous composition. Examples of the aqueous composition include, but are not limited to, saline, phosphate buffered saline (PBS) and acidic aqueous solution (e.g., sulfate acid aqueous solution, hydrochloric acid aqueous solution, phosphoric acid aqueous solution, acetic acid aqueous solution, citric acid aqueous solution). Preferably, the acidic aqueous solution is an acidic aqueous solution in compliance with food grade requirements, such as aqueous solution of sulfuric acid of food grade, aqueous solution of hydrochloric acid of food grade, aqueous solution of phosphoric acid of food grade, and the like. In some embodiments, the amino acid-chelated zinc is mixed with an acidic aqueous solution to provide an aqueous composition with a pH less than 7, such as pH 1, pH 1.5, pH 2, pH 2.5, pH 3, pH 3.5, pH 4, pH 4.5, pH 5, pH 5.5, pH 6, pH 6.5 or 6.9 or within a range between any two of the values described herein.

Optionally, the pH can be adjusted depending on the requirement(s) for practical use of the composition, such as subjects to be administered, administration routes, etc. For example, when the composition is administered to a human or a mouse/rat via oral administration, the composition can be an aqueous composition with a pH of 2; when the composition is administered to a mature pig, the composition can be an aqueous composition with a pH of 3.5; and, when the composition is administered to a sucking pig, the composition can be an aqueous composition with a pH of 4.5.

The amount of amino acid-chelated zinc in the composition provided in accordance with the present invention can be determined based on the daily zinc intake required by the subject(s) to be administered. In general, the daily zinc intake for a healthy adult female is 12 mg, the daily zinc intake for a healthy adult male is 15 mg, and the upper limit of the daily zinc intake is 35 mg. In addition, the amount of amino acid-chelated zinc in the composition can also be optionally adjusted depending on desired administration frequencies, such as once a day, multiple times a day, or once every few days.

In some embodiments of the composition provided in accordance with the present invention, the composition is a pharmaceutical composition, and the pharmaceutical composition can be used for treating an allergic disease, treating an autoimmune disease and/or preventing an organ transplant rejection, or can be used for alleviating discomfort resulted from the aforementioned disease(s). Examples of the allergic disease include, but are not limited to, allergic rhinitis, allergic conjunctivitis, atopic dermatitis, urticaria, allergic gastroenteritis and asthma. Examples of the autoimmune disease include, but are not limited to, rheumatoid arthritis, type 1 diabetes, multiple sclerosis, myasthenia gravis, systemic lupus erythematosus and ankylosing spondylitis.

The pharmaceutical composition provided in accordance with the present invention can be administered systemically or topically and can be delivered by various drug delivery system (DDSs). Suitable drug delivery systems include, but are not limited to, an oral drug delivery system and an injectable drug delivery system. For example, the pharmaceutical composition can be administered to a subject in need by one or more of the following administration routes: oral administration, intravenous injection (including drip infusion and bolus injection), intramuscular injection, subcutaneous injection, arterial injection or intraperitoneal injections, but is not limited thereby. In addition, to enhance bioavailability, control drug release speed, target the lesion precisely and reduce side effects, the pharmaceutical composition provided in accordance with the present invention can be delivered by liposomes, microcapsules, nanoparticles, and the like.

The pharmaceutical composition provided in accordance with the present invention can be in any suitable form without special limitation, and can be presented in an appropriate dosage form depending on the desired purpose(s). For example, the pharmaceutical composition can be available as a suspension, an emulsion, a solution (such as syrup, elixirs, tincture, etc.), coated tablets, tablets, granules, powders, capsules, pellets, pills and an injection, but is not limited thereby. Depending on the form and purpose(s), a pharmaceutically acceptable excipient can be chosen to provide the pharmaceutical composition. Suitable excipients are well known by a person skilled in the art of pharmaceutical manufacturing and include, but are not limited to, a diluent, a surfactant, a flow agent, a disintegrating agent, an adhesive, a buffer, a colorant, a flavoring agent, an antioxidant, a preservative and a film forming agent.

In some embodiments of the composition provided in accordance with the present invention, the composition can be a food composition, and the food composition can be used for immune regulation. The food composition can be a beverage, a solid food or a semi-solid food, and can be provided in a form of a health food, a daily supplement, a functional food, a nutritional supplement or a special nutrition food. In some embodiments, the food composition is a health food.

Depending on the form and purpose(s), the food composition in accordance with the present invention can comprise any suitable food additive(s). Examples of the food additive include, but are not limited to, a preservative, a bactericide, an antioxidant, a bleaching agent, a color fasting agent, a swelling agent, a nutrition additive, a colorant, a flavoring agent (e.g., a sweetener), a pasting agent, a binding agent, chemicals for food industry, an emulsifier, and agents for food quality improvement, fermentation and food processing.

### Method for Regulating Immunity and Inhibiting Dendritic Cell Maturation

In another aspect of the present invention, a method for regulating immunity and/or inhibiting dendritic cell maturation is provided, wherein the method comprises administering an effective amount of the composition described above to a subject in need.

In the method for regulating immunity and/or inhibiting dendritic cell maturation in accordance with the present invention, wherein the subject in need refers to a subject with immune imbalance (especially abnormal immune activation), such as patients with autoimmune diseases, or a subject who has undergone an organ transplant.

### Combination for Preparing a Tolerogenic Dendritic Cell

In still another aspect of the present invention, provided is a combination, which comprises: (1) an amino acid-chelated zinc; and (2) a dendritic cell medium. Furthermore, the combination can be used for preparing a tolerogenic DC, and the amino acid-chelated is defined as above.

In the combination provided in accordance with the present invention, there is no specific limitation on the dendritic cell medium as long as the nutrient(s) in the dendritic cell medium is sufficient to cultivate a dendritic cell. In some embodiments, the dendritic cell medium is RPMI-1640 medium comprising interleukin-4 (IL-4), granulocyte-macrophage colony-stimulating factor (GM-CSF) and fetal bovine serum (FBS).

In some embodiments of the combination provided in accordance with the present invention, the combination further comprises a dendritic cell, and the dendritic cell is preferably an immature dendritic cell.

In some embodiments of the combination provided in accordance with the present invention, the amino acid-chelated zinc has an amount of zinc ranging from 1 mg to 25 mg per mL of the dendritic cell medium. For example, the amino acid-chelated zinc has an amount of zinc is 1 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 10.5 mg, 11 mg, 11.5 mg, 12 mg, 12.5 mg, 13 mg, 13.5 mg, 14 mg, 14.5 mg, 15 mg, 15.5 mg, 16 mg, 16.5 mg, 17 mg, 17.5 mg, 18 mg, 18.5 mg, 19 mg, 19.5 mg, 20 mg, 20.5 mg, 21.5 mg, 22 mg, 22.5 mg, 23 mg, 23.5 mg, 24 mg, 24.5 mg or 25 mg per mL of the dendritic cell medium, or within a range between any two of the values described herein.

The combination provided in accordance with the present invention can be a kit or a composition. When the combination provided in accordance with the present invention is a kit, the component (1), amino acid-chelated zinc, and the component (2), dendritic cell medium, are usually packaged separately and stored independently in different containers (e.g., a plastic bag, a plastic bottle, a glass bottle, an ampoule), and can be transported or sold either alone or in combination as a set. The kit can further comprise an instruction manual, which provides the procedures and program for the user to mix the components on-site for cell culture, processing and application.

When the component (1) and the component (2) are packaged separately and stored independently in different containers, they can be stored at different environments. For example, the container of the component (1) can be stored at room temperature or a temperature of 0° C. to 7° C. or −20° C.; the container of the component (2) can be stored at a temperature of 0° C. to 7° C.

In addition, when the combination provided in accordance with the present invention is a composition, the component of (1), amino acid-chelated zinc, and the component (2), dendritic cell medium, are usually mixed and stored in a same container (e.g., a plastic bag, a plastic bottle, a glass bottle, an ampoule).

When the combination provided in accordance with the present invention further comprises a dendritic cell, the dendritic cell can be stored in a cell storage container known in the art (e.g., a cryotube). In some embodiments, the dendritic cell is mixed with a cryoprotective agent and placed into a cryotube to store at −80° C. or in liquid nitrogen. The cryoprotective agent is well known in the art, such as a cell medium containing 5% to 10% by volume of Dimethyl sulfoxide (DMSO), or a commercially available cryoprotective agent.

### Method for Preparing a Tolerogenic Dendritic Cell

In still another aspect of the present invention, a method for preparing a tolerogenic dendritic cell is provided, wherein the method comprises contacting a dendritic cell with an effective amount of an amino acid-chelated zinc.

In some embodiments, the contact is performed by culturing the dendritic cell in a dendritic cell medium containing the amino acid-chelated zinc. Furthermore, the amino acid-chelated zinc, dendritic cell and dendritic cell medium are defined as above.

The present invention will be further illustrated in detail with specific examples as follows. However, the following examples are provided only for illustrating the present invention and the scope of the present invention is not limited thereby. The scope of the present invention will be indicated in the appended claims.

### EXAMPLES

The materials and equipment as used in the following examples are listed as below.
1. Zinc sulfate: purchased from SIGMA Co., product no.: 1282101001.
2. PBS: purchased from Corning Co., product no.: 21-040-CM.
3. Mouse: purchased from BioLASCO Taiwan Co., Ltd., strain: C57BL/6.
4. Lewis rats: purchased from BioLASCO Taiwan Co., Ltd., 7 week-old, female.
5. RPMI-1640 medium: purchased from Thermo Fisher Co., product no.: 11875093.
6. Red blood cell lysis buffer (RBC lysis buffer): purchased from SIGMA Co., product no.: R7757.
7. Dendritic cell medium (DC medium): RPIM-1640 medium containing 500 U/mL of IL-4 (purchased from Thermo Fisher Co., product no.: 214-14), 1000 U/mL of GM-CSF (purchased from Thermo Fisher Co., product no.: 315-03) and 10% (v/v) of FBS (purchased from Thermo Fisher Co., product no.: A3160402).
8. Flow cytometry: CytoFLEX S, Beckman.
9. Collagen: purchased from Chondrex Co., Immunization Grade Bovine Type II Collagen.
10. Freund's Incomplete Adjuvant (IFA): purchased from SIGMA Co.
11. Interferon-γ (IFN-γ): purchased from Thermo Fisher Co., product no.: 315-05.
12 R848 (resiquimod): purchased from MCE Co., product no.: HY-13740.
13. Polyinosinic-Polycytidylic acid (poly I:C): purchased from MCE Co., product no.: HY-107202.
14. Lipopolysaccharide (LPS): purchased from SIGMA Co., product no.: L2630.
15. Analytical reagents and antibodies for flow cytometry: FcR Blocking Reagent (purchased from MACS Co., product no.: 130-092-575), CD11c antibody (purchased from biolegend Co., product no.: 117309), CD40 antibody (purchased from biolegend Co., product no.: 124607), CD80 antibody (purchased from biolegend Co., product no.: 104705), CD83 antibody (purchased from biolegend Co., product no.: 121509), CD86 antibody (purchased from biolegend Co., product no.: 105031), MHC II antibody (purchased from biolegend Co., product no.: 107631), isotype control antibody (purchased from biolegend Co., product no.: 400905, 400411, 400535).
16. ELISA analysis for IL12P70: purchased from Arigo Co., product no.: ARG80201, including a 96-well plate pre-coating IL12P70 antibodies, a IL12P70 standard, a wash buffer, antibody conjugates, a HRP-Streptavidin solution, a TMB Reagent and a Stop Solution. Before using, each reagent/solution is placed at room temperature for 20 minutes.
17. Methotrexate (MTX): purchased from MCE Co., product no.: HY-14519

### Preparation Example

### A. Preparation of Amino Acid-Chelated Zinc

The amino acid-chelated zinc used in the examples is prepared by the following method: sieving an aqueous solution of zinc sulfuric acid of food grade and a glycine of food grade with a mesh sieve, respectively, and mixing them at a molar ratio ranging from 1:1 to 1:4; refrigerating the mixture thus obtained at 0° C. to 4° C. for 12 hours to 24 hours; and, heating the refrigerated mixture at a temperature of 80° C. for 12 hours to obtain a glycine-chelated zinc powder.

For the cell experimentation of Example 1, the glycine-chelated zinc powder was adjusted with the DC medium to form solutions at a zinc concentration of 13.08 mg/mL, 6.54 mg/mL or 3.27 mg/mL, and for the animal experimentation of Example 2, the glycine-chelated zinc powder was dissolved into 2 mL of PBS to obtain a solution containing 0.075 mg zinc (hereinafter referred to as “CMC-Zn”). In addition, the pH values of the aforesaid CMC-Zn at different concentrations of zinc were adjusted with citric acid to 2, respectively (hereinafter referred to as “modified CMC-Zn”).

### B. Preparation of Mouse Bone Marrow Dendritic Cells (BMDCs)

Mice were sacrificed with carbon dioxide and immersed in 75% alcohol. After immersing for 3 minutes, the mice were removed from the alcohol and wiped. Then, the mouse limbs were fixed on a Styrofoam board. Next, the entire femur and tibia were collected by carefully cutting off the mouse skin and muscle. A filter (100 μm) was placed in a 3-cm dish and 2 ml of RPMI-1640 medium was added into the dish. Two ends of the femur were cut, and the bone marrow was collected by repeatedly washing the femur with the cell medium using a 26 G syringe until the femur appeared white. The bone marrow in the tibia was also collected by the same method. The filter was scraped by the reverse side of 1 mL tip to break up the cell clumps in the bone marrow and washed with the cell medium in the dish several times. Then, the cell medium in the dish was collected and placed into a tube to centrifuge at 1500 rpm for 3 minutes. After centrifuging, the supernatant was removed and 1 mL of RBC lysis buffer was added into the tube. After standing for 5 minutes, the tube was centrifuged at 1500 rpm for 3 minutes again. After removing the supernatant, a milky-white pellet was obtained. The pellet was re-suspended with the DC medium and the cell number of the suspension thus obtained was counted.

The cell concentration of the suspension was adjusted to 5×10⁵ cells per mL. 1 mL of the suspension was added into each well of a 24-well plate. The 24-well plate was incubated at 37.5° C. and 5% CO₂ for 2 days. The unattached granulocytes were removed by carefully removing the cell medium, and then, 0.5 mL of DC medium was added into each well of the 24-well plate. After incubating 3 days, the medium in each well was replaced with 0.5 mL of fresh DC medium and continuously incubated for 3 days. The 24-well plate was taken out from the incubator, and the medium in each well was carefully removed. The bottom of each well was gently pipetted with 2 mL of PBS several times. The pipetted PBS was collected into a centrifuge tube to count the number of cells and analyze the expression level of CD11c using flow cytometry.

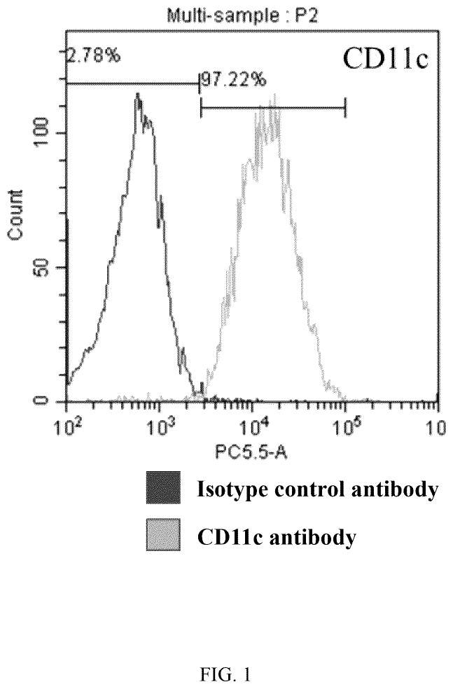

As shown in FIG. 1, the expression level of CD11c in the resulting cells is more than 70% as analyzed by flow cytometry, and thus the cells are served as BMDCs for subsequent experimentations.

C. Preparation of Rats with Rheumatoid Arthritis

Lewis rats were bred under the condition with a temperature of 21° C. to 25° C., a humidity level of 40% to 70%, and a lighting period of 12 hours (7 a.m. to 7 p.m.). The rats had free access to the feed. All the rats were monitored for at least 6 days to ensure their health to prepare a collagen-induced arthritis (CIA) animal model.

A collagen emulsion was prepared by evenly mixing collagen and IFA at a volume ratio of 1:1 using a homogenizer in a cold water bath. On Day 1, 200 μL of the collagen emulsion was injected subcutaneously at the base of the rat's tail, followed by a booster injection of 100 μL of the collagen emulsion at a different site on the base of the tail on Day 8. All the rats successfully developed CIA by Day 17 (hereinafter referred to as “CIA rat”).

### Example 1: Effect of Amino Acid-Chelated Zinc on Dendritic Cell Maturation

The secretion level of IL12P70 in immature DCs is very low, whereas mature DCs secret a large amount of IL12P70, allowing it an indicator of DC maturation. In this example, the expression levels of the surface markers (CD11c, CD40, CD83, CD86) on the BMDCs were analyzed using flow cytometry, and the secretion level of IL12p70 in the BMDCs' medium was measured with an ELISA kit, to observe the effects of amino acid-chelated zinc on dendritic cell maturation.

### (1-1) Cell Treatment

The BMDCs obtained from Preparation example B were seeded to a 24-well plate at a concentration of 1×10⁵ cells/well, and the cells in each group were cultured with 500 μL of the following medium at 37° C. and 5% CO₂ for 48 hours:
(1) “Immature DC” group: DC medium;
(2) “Mature DC” group: DC medium containing 1000 U/mL of IFN-γ, 2.5 μg/mL of R848, 20 μg/mL of poly I:C and 10 ng/ml of LPS;
(3) “Zinc sulfate (low concentration)” group: DC medium containing zinc sulfate at a zinc concentration of 3.27 mg/mL;
(4) “Zinc sulfate (medium concentration)” group: DC medium containing zinc sulfate at a zinc concentration of 6.54 mg/mL;
(5) “Zinc sulfate (high concentration)” group: DC medium containing zinc sulfate at a zinc concentration of 13.08 mg/mL;
(6) “CMC-Zn (low concentration)” group: the CMC-Zn at a zinc concentration of 3.27 mg/mL provided by Preparation example A;
(7) “CMC-Zn (medium concentration)” group: the CMC-Zn at a zinc concentration of 6.54 mg/mL provided by Preparation example A;
(8) “CMC-Zn (high concentration)” group: the CMC-Zn at a zinc concentration of 13.08 mg/mL provided by Preparation example A;
(9) “Modified CMC-Zn (low concentration)” group: the modified CMC-Zn at a zinc concentration of 3.27 mg/mL provided by Preparation example A;
(10) “Modified CMC-Zn (medium concentration)” group: the modified CMC-Zn at a zinc concentration of 6.54 mg/mL provided by Preparation example A;
(11) “Modified CMC-Zn (high concentration)” group: the modified CMC-Zn at a zinc concentration of 13.08 mg/mL provided by Preparation example A;

After culturing for 48 hours, the cells and their medium in each group were collected to conduct the following analysis, respectively.

### (1-2) Flow Cytometry Analysis

The cells obtained from (1-1) were counted and the cell concentration was adjusted with PBS to provide a cell suspension of 1×10⁵ cells to 5×10⁵ cells per 100 μL. 100 μL of the cell suspension was evenly mixed with 20 μL of FcR Blocking Reagent, and the mixture thus obtained was placed at 4° C. for 10 minutes. Furthermore, the antibodies of CD11c, CD40, CD80, CD83, CD86, MHC II and corresponding isotype control were added into the tubes containing the mixture, respectively. After vortexing for 1 second, the tubes were placed at room temperature in the dark for 15 minutes. Then, 300 μL of PBS was added into each tube, and after vortexing for 1 second, the tubes were centrifuged at 200×g for 5 minutes. The resulting supernatants were removed and the cells were re-suspended with 300 μL of PBS. Finally, the cell suspensions thus obtained were analyzed by flow cytometry, wherein all the cell suspension of each group were kept on ice in the dark until the analysis was conducted.

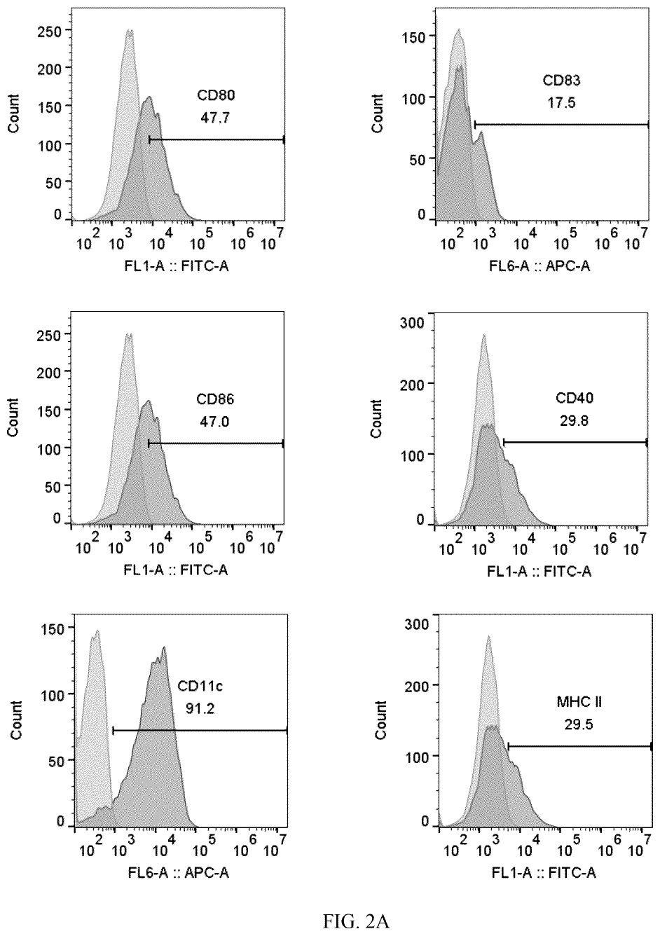

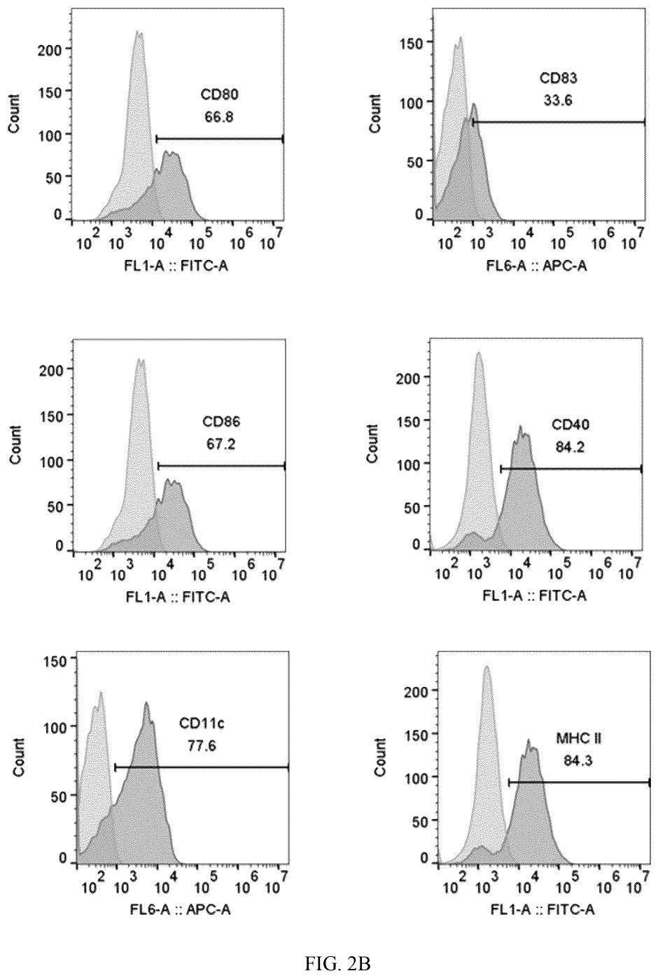

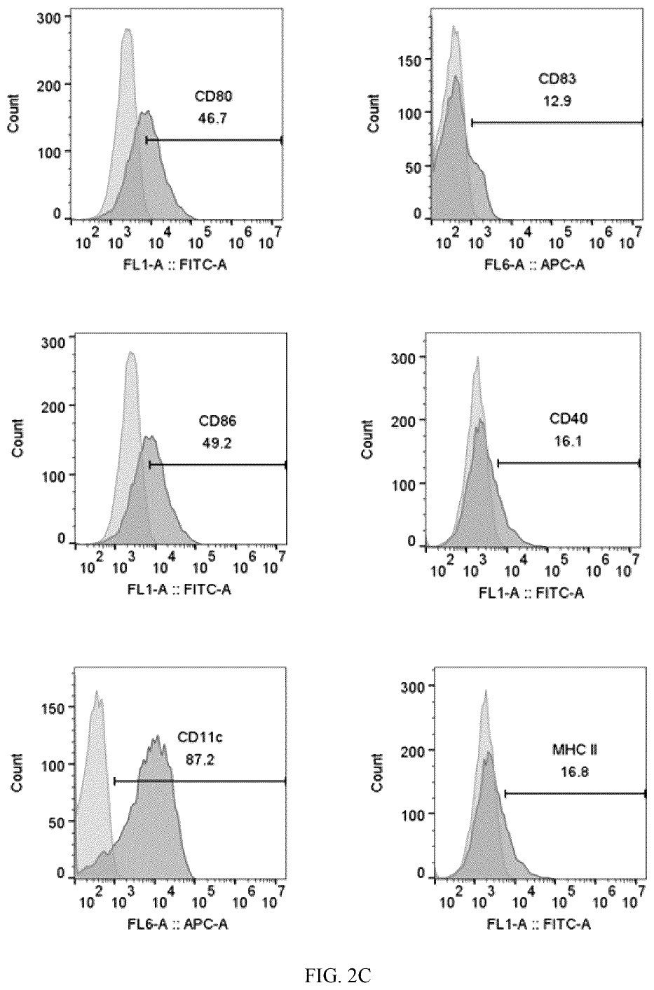

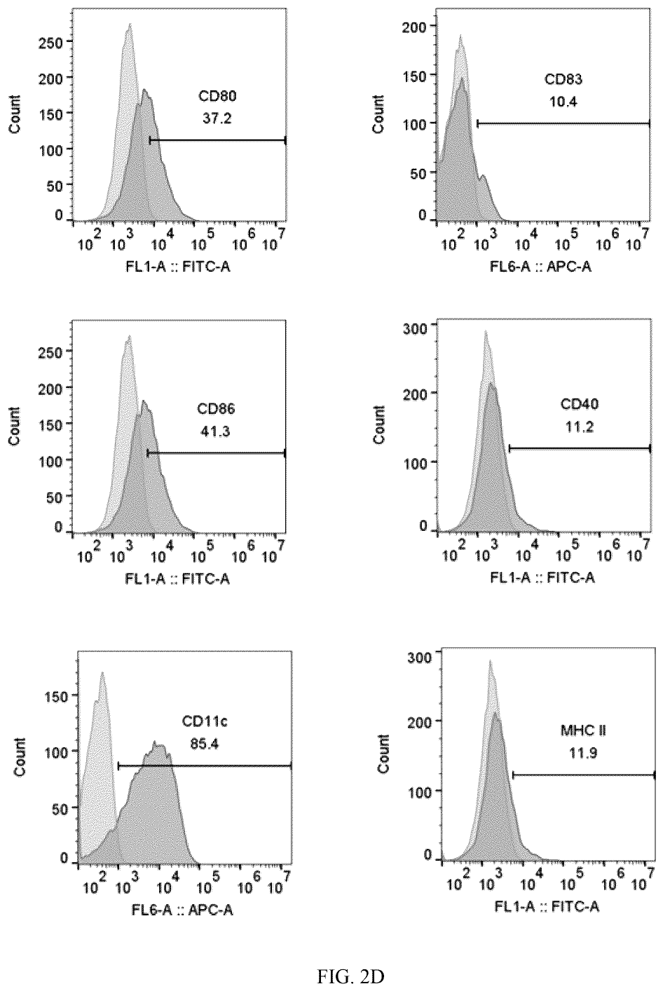

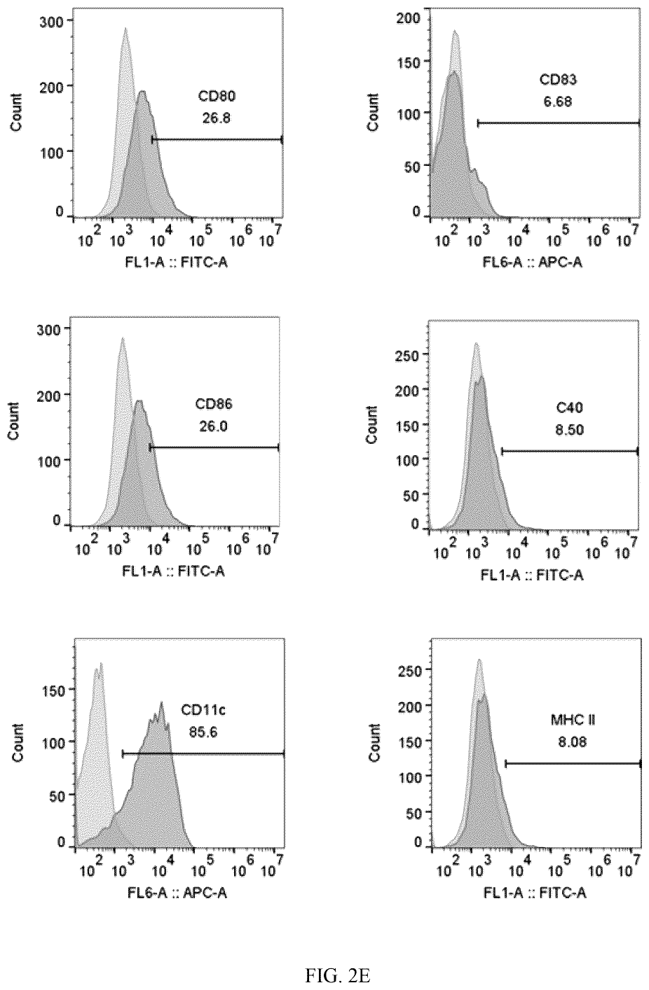

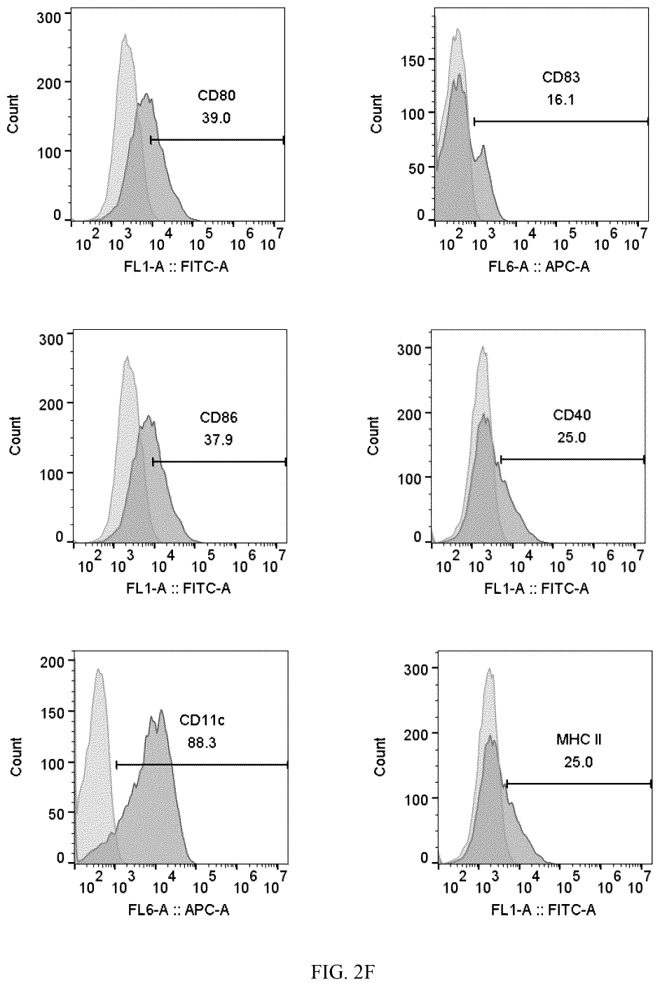

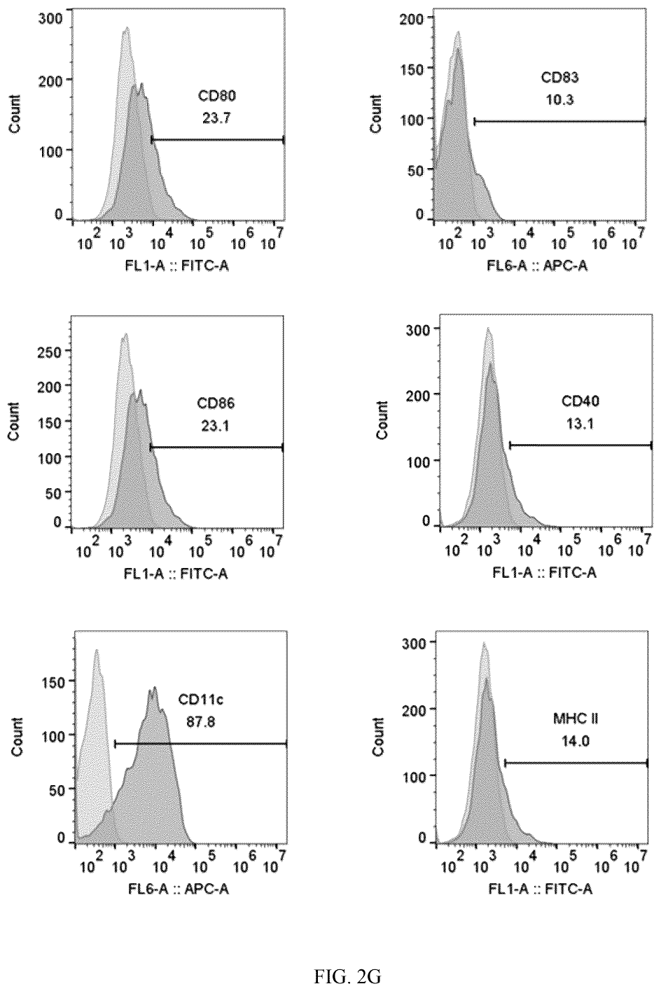

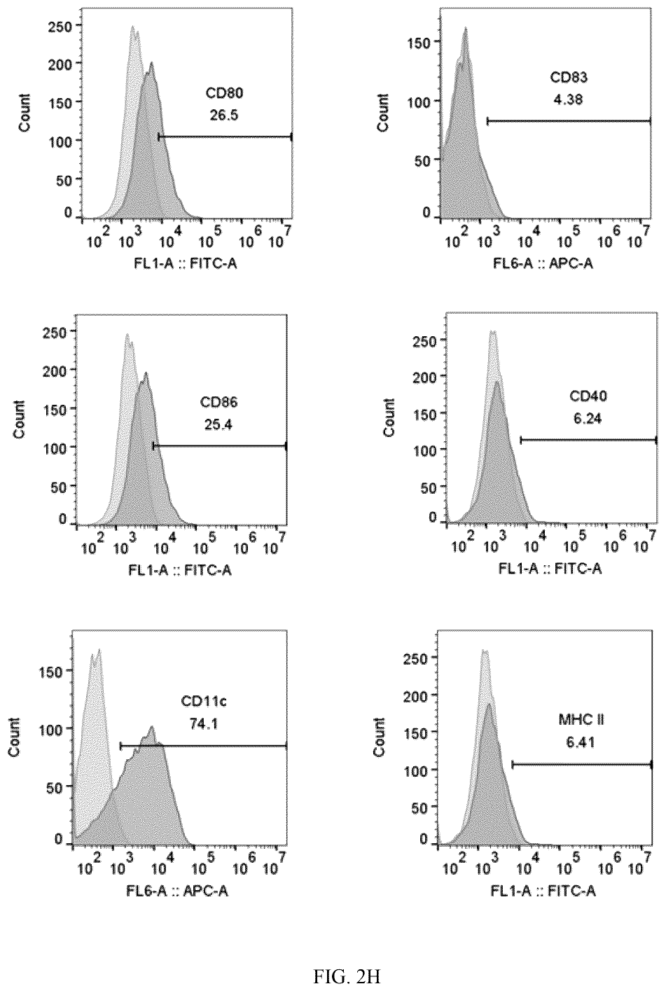

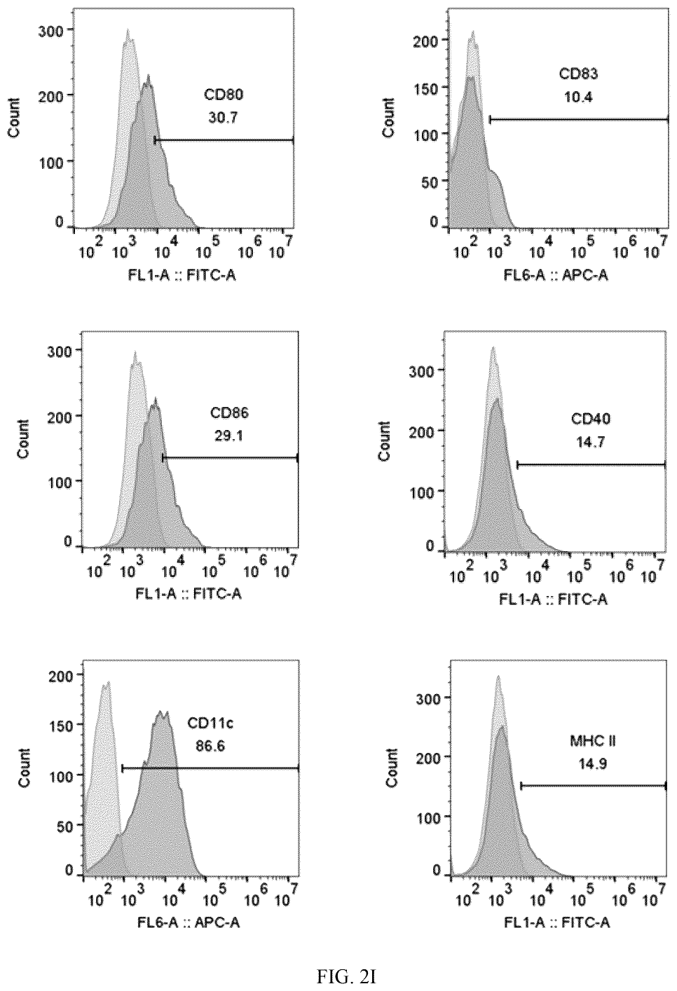

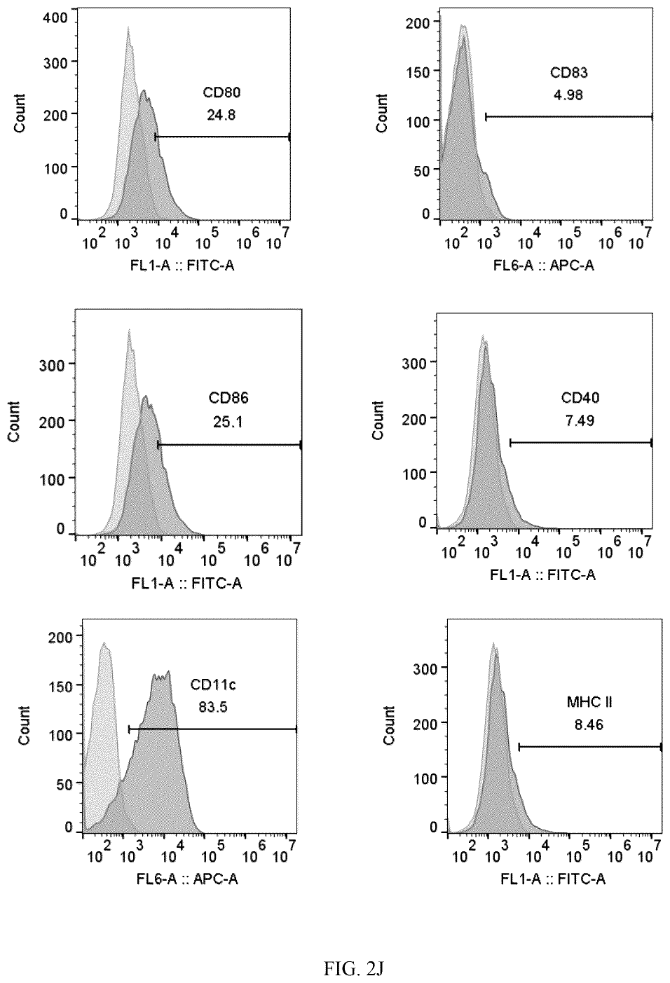

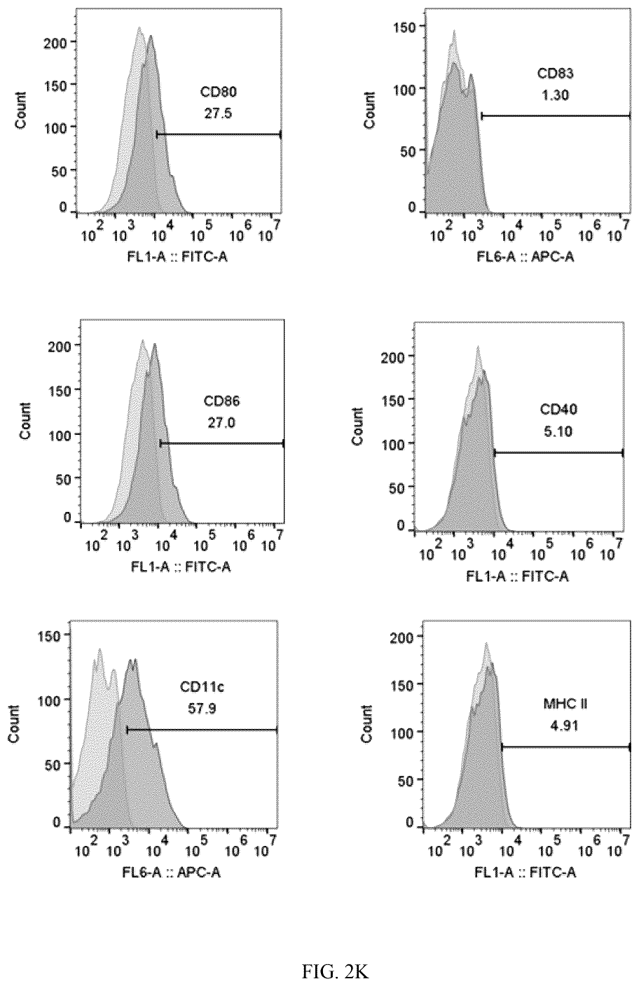

As shown in Table 1 and FIGS. 2A to 2K, the induced mature BMDCs and/or the BMDCs treated with zinc sulfate, CMC-Zn or modified CMC-Zn were analyzed by flow cytometry to determine the expression levels of the dendritic cells maturation marker (CD40, CD80, CD83, CD86 and MHC II). The analysis results showed that the expression levels of the markers on the induced mature BMDCs were all increased, indicating that the study platform was reliable. All the groups of zinc sulfate, CMC-Zn and modified CMC-Zn demonstrated the ability to inhibit dendritic cell maturation, and the inhibitory ability was directly proportional to the zinc concentration. CMC-Zn and modified CMC-Zn at a low concentration were effective in inhibiting dendritic cell maturation, and the expression levels of the markers in the “CMC-Zn (low concentration)” group and the “Modified CMC-Zn (low concentration)” group were even lower than those of the “Immature DC” group. As compared to the groups of zinc sulfate, the expression levels of CD80 and CD86 in the groups of CMC-Zn and modified CMC-Zn at both low and medium concentration were significantly decreased, indicating that the abilities of CMC-Zn and modified CMC-Zn to inhibit dendritic cell maturation were better than that of zinc sulfate. The aforesaid results demonstrated that the amino acid-chelated zinc was effective in inhibiting dendritic cell maturation, and thus could be used for immune regulation (e.g., treating an allergic disease, treating an autoimmune disease, preventing an organ transplant rejection) and the preparation of tolerogenic DCs that could serve as an immunosuppressant.

TABLE 1

| Groups | CD11c | CD40 | CD80 | CD83 | CD86 | MHC II |
|---|---|---|---|---|---|---|
| Immature DC | 91 | 29.8 | 47.7 | 17.6 | 47 | 29 |
| Mature DC | 77.6 | 84.2 | 66.8 | 33.6 | 67.2 | 84.3 |
| Zinc sulfate | 87.2 | 16.1 | 46.7 | 12.9 | 49.2 | 16.8 |
| (low concentration) | | | | | | |
| Zinc sulfate | 85.4 | 11.2 | 37.2 | 10.4 | 41.3 | 11.9 |
| (medium concentration) | | | | | | |
| Zinc sulfate | 85.6 | 8.5 | 26.8 | 6.68 | 26 | 8.08 |
| (high concentration) | | | | | | |
| CMC-Zn | 88.3 | 25 | 39 | 16.1 | 37.9 | 25 |
| (low concentration) | | | | | | |
| CMC-Zn | 87.8 | 13.1 | 23.7 | 10.3 | 23.1 | 14 |
| (medium concentration) | | | | | | |
| CMC-Zn | 74.1 | 6.24 | 26.5 | 4.38 | 25.4 | 6.41 |
| (high concentration) | | | | | | |
| Modified CMC-Zn | 86.6 | 14.7 | 30.7 | 10.4 | 29.1 | 14.9 |
| (low concentration) | | | | | | |
| Modified CMC-Zn | 83.5 | 7.49 | 24.8 | 4.98 | 25.1 | 8.46 |
| (medium concentration) | | | | | | |
| Modified CMC-Zn | 57.9 | 5.1 | 27.5 | 1.3 | 27 | 4.91 |
| (high concentration) | | | | | | |

### (1-3) ELISA Analysis

The media obtained from each group were placed into tubes, respectively, and the tubes were centrifuged (400×g) at 4° C. for 10 minutes to collect the supernatants. The IL12p70 standard was subjected to a serial dilution to provide a standard curve. The standards at various concentrations and the supernatants obtained from each group were added into a 96-well plate pre-coating with IL12p70 antibodies (100 μL per well, in duplicate). The 96-well plate was gently tapped to ensure the liquid in each well was evenly mixed, and placed in a 37° C. incubator for 1.5 hours. Furthermore, 350 μL of 1× wash buffer was added into each well of the plate, and after soaking for 30 seconds, the liquid in each well was removed. The washing step was repeated fifth times. After washing, 100 μL of 1× antibody conjugates was added into each well, and the 96-well plate was gently tapped to ensure the liquid in each well was evenly mixed. The 96-well plate was placed in the 37° C. incubator for 1 hour. Again, the washing step was repeated fifth times by using the 1× wash buffer. Then, 100 μL of 1× HPR-Streptavidin solution was added into each well, and after gently tapping, the 96-well plate was placed in the 37° C. incubator in the dark. After 30 minutes, the washing step was repeated fifth times again. Thereafter, 100 μL of TMB reagent was added into each well, and after gently tapping, the 96-well plate was placed in the 37° C. incubator in the dark for 15 minutes. The 96-well plate was removed from the incubator, and 100 μL of Stop Solution was added into each well. The absorbance value at 450 nm (OD₄₅₀) in each well was detected within 3 minutes. Based on the standard curve resulted from the absorbance values of IL12p70 standards at various concentrations, the IL12p70 amounts corresponding to the absorbance values of each group were calculated as shown in Table 2.

As shown in Table 2, expect for the medium of the “Mature DC” group, IL12P70 was not detected in the media obtained from the “Immature DC” group and the groups of zinc sulfate, CMC-Zn and modified CMC-Zn. These results also demonstrated that the amino acid-chelated zinc was effective in inhibiting dendritic cell maturation, and thus could be used for immune regulation (e.g., treating an allergic disease, treating an autoimmune disease, preventing an organ transplant rejection) and the preparation of tolerogenic DCs that could serve as an immunosuppressant.

TABLE 2

| Groups | IL12P70 amount (pg/mL) |
|---|---|
| Immature DC | ND |
| Mature DC | 113.06 |
| Zinc sulfate | ND |
| (low concentration) | |
| Zinc sulfate | ND |
| (medium concentration) | |
| Zinc sulfate | ND |
| (high concentration) | |
| Modified CMC-Zn | ND |
| (low concentration) | |
| Modified CMC-Zn | ND |
| (medium concentration) | |
| Modified CMC-Zn | ND |
| (high concentration) | |
| Modified CMC-Zn | ND |
| (low concentration) | |
| Modified CMC-Zn | ND |
| (medium concentration) | |
| Modified CMC-Zn | ND |
| (high concentration) | |

*ND: not detected

### Example 2: Effect of Amino Acid-Chelated Zinc on Autoimmune Disease

Rheumatoid arthritis is an autoimmune disease, and the patient's immune system is abnormally activated to attack its own cells and tissues, thereby causing a long-term chronic inflammation in the body. In this example, the therapeutic effects of amino acid-chelated zinc on autoimmune diseases (e.g., rheumatoid arthritis) were observed by a CIA animal model.

As described in Preparation example C, all the rats successfully developed CIA by Day 17. Therefore, the CIA rats were randomly divided into four groups from Day 17 to administer the following solutions until the rats were sacrificed on Day 24, wherein the healthy rats not to induce arthritis were served as the control group:
(1) Control group (five healthy rats): 2 mL of PBS;
(2) CIA group (five CIA rats): 2 mL of PBS;
(3) MTX group (three CIA rats): 1 mg of MTX per kilogram of rat, dissolved in 2 mL of PBS;
(4) CMC-Zn group (five CIA rats): CMC-Zn at a zinc concentration of 0.075 mg, dissolved in 2 mL of PBS; and
(5) Modified CMC-Zn group (five CIA rats): modified CMC-Zn at a zinc concentration of 0.075 mg, dissolved in 2 mL of PBS.

The rat's paws were observed and their paw volumes were measured on Days 0, 3, 7, 10, 14, 17, 21 and 24. The arthritis severity of rats was evaluated by using the qualitative scoring system of Table 3, and the paw volume was determined by the following approach in accordance with the buoyancy principle of Archimedes' law: adding an appropriate amount of water into a beaker; placing the beaker on a scale and resetting to zero; placing the rat's paw into the water and immersing it to the target depth; and recoding the reading once it had stabilized.

TABLE 3

| Degrees of severity | Score |
|---|---|
| Normal | 0 |
| The ankle or wrist has mild but definite redness | 1 |
| and swelling; or the individual finger has | |
| noticeable redness and swelling (regardless of | |
| the number of fingers). | |
| The ankle or wrist has moderate redness and | 2 |
| swelling. | |
| The whole paw (including the fingers) has | 3 |
| severe redness and swelling. | |
| The extreme inflammation involves multiple | 4 |
| joints. | |

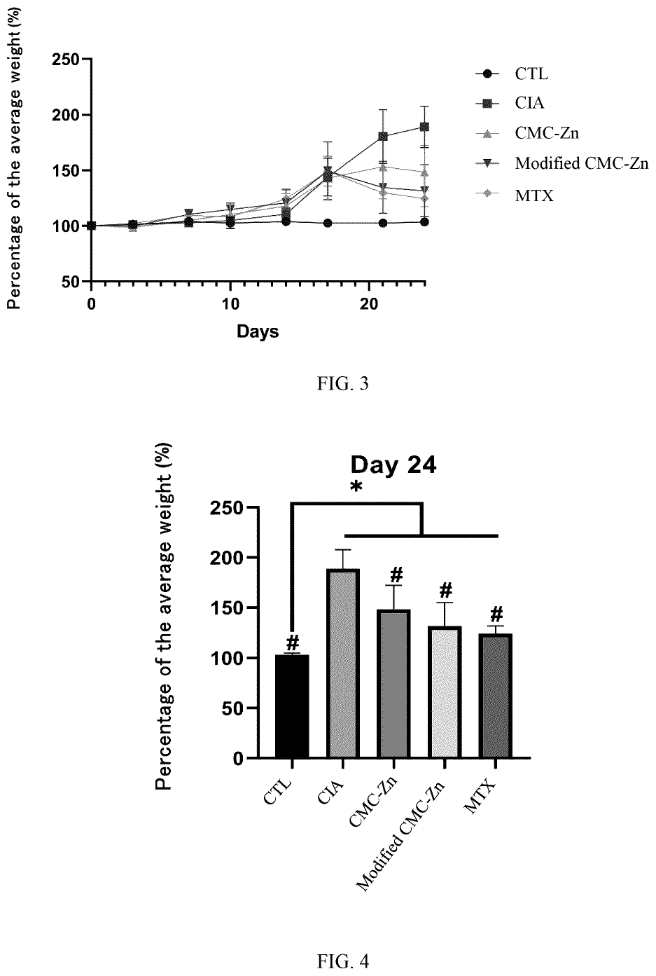

As shown in FIG. 3, expect for the paws of the healthy rats in the control group, all the weight percentages of the CIA rats' paws were markedly increased, indicating that the CIA rats all successfully developed arthritis. As compared to the rats of the CIA group, the weight percentages of the rats' paws in the MTX group, CMC-Zn group and Modified CMC-Zn group were all gradually decreased after administering MTX, CMC-Zn or modified CMC-Zn, respectively. This indicated that the arthritis symptoms of the CIA rats administered with MTX, CMC-Zn or modified CMC-Zn were gradually improved, wherein the effect of modified CMC-Zn was even comparable to that of MTX. FIG. 4 showed that on Day 24, as compared to the rats of the control group, the weight percentage of the CIA rats' paws were all significantly increased, and as compared to the rats of the untreated CIA rats, the weight percentages of the rats' paws in the MTX group, CMC-Zn group and Modified CMC-Zn group were all significantly decreased. The aforesaid results showed that the administration of the amino acid-chelated zinc to a subject was effective in alleviating the symptoms of rheumatoid arthritis, and thus, the amino acid-chelated zinc could be used for treating autoimmune disease (e.g., rheumatoid arthritis).

Table 4 showed the average weight percentages of both paws on Days 0, 3, 7, 10, 14, 17, 21 and 24, which were obtained by scoring the arthritis severity of rats in each group according to the criteria listed in Table 3. As compared to the CIA group, the rats in each of the MTX group, CMC-Zn group and Modified CMC-Zn group showed slower progression or even reduction in arthritis severity after Day 17 (i.e., after administering MTX, CMC-Zn or modified CMC-Zn). Especially, the score of arthritis severity of rats in the Modified CMC-Zn group was even lower than that of the MTX group.

TABLE 4

| | Day 0 | Day 3 | Day 7 | Day 10 | Day 14 | Day 17 | Day 21 | Day 24 |
|---|---|---|---|---|---|---|---|---|
| Control | 0 | 0 | 0 | 0 | 0 | 0 | 0 | 0 |
| group | | | | | | | | |
| CIA | 0 | 0 | 0 | 0 | 0.8 | 3.6 | 6.0 | 6.6 |
| group | | | | | | | | |
| MTX | 0 | 0 | 0 | 0 | 0.3 | 3.7 | 3.7 | 3 |
| group | | | | | | | | |
| CMC-Zn | 0 | 0 | 0 | 0 | 0.4 | 3.8 | 4.4 | 4.6 |
| group | | | | | | | | |
| Modified | 0 | 0 | 0 | 0 | 1 | 4.2 | 2 | 1.8 |
| CMC-Zn | | | | | | | | |
| group | | | | | | | | |

As can be seen from the aforementioned results, the amino acid-chelated zinc is not only effective in inhibiting dendritic cell maturation, but also effective in alleviating the symptoms in the subjects with rheumatoid arthritis. Therefore, the amino acid-chelated zinc can be used for immune regulation (e.g., treating an allergic disease, treating an autoimmune disease, preventing an organ transplant rejection), and preparation of tolerogenic DCs that can serve as an immunosuppressant.

The above embodiments are merely to explain the principles and effects of the present invention and to describe its technical features, and are not intended to limit the scope of the present invention. Any modifications or arrangements that a person skilled in this field can easily achieve without deviating from the principle of the present invention fall within the scope claimed by the present invention. Therefore, the scope of protection of the present invention is defined by the claims appended hereto.

What is claimed is:

1. A method for regulating immunity in a subject in need, comprising administering to the subject a composition, wherein the composition comprises an effective amount of an amino acid-chelated zinc.

2. The method of claim 1, wherein the composition is an aqueous composition with a pH less than 7.

3. The method of claim 1, wherein the amino acid-chelated zinc has a molar ratio of zinc and amino acid ranging from 1:1 to 1:10.

4. The method of claim 1, wherein the amino acid-chelated zinc is selected from the group consisting of glycine-chelated zinc, alanine-chelated zinc, valine-chelated zinc, leucine-chelated zinc, isoleucine-chelated zinc and combinations thereof.

5. The method of claim 1, which is for treating an allergic disease, treating an autoimmune disease and/or preventing an organ transplant rejection.

6. The method of claim 2, which is for treating an allergic disease, treating an autoimmune disease and/or preventing an organ transplant rejection.

7. The method of claim 3, which is for treating an allergic disease, treating an autoimmune disease and/or preventing an organ transplant rejection.

8. The method of claim 4, which is for treating an allergic disease, treating an autoimmune disease and/or preventing an organ transplant rejection.

9. A method for inhibiting dendritic cell maturation in a subject in need, comprising administering to the subject a composition, wherein the composition comprises an effective amount of an amino acid-chelated zinc.

10. The method of claim 9, wherein the composition is an aqueous composition with a pH less than 7.

11. The method of claim 9, wherein the amino acid-chelated zinc has a molar ratio of zinc and amino acid ranging from 1:1 to 1:10.

12. The method of claim 9, wherein the amino acid-chelated zinc is selected from the group consisting of glycine-chelated zinc, alanine-chelated zinc, valine-chelated zinc, leucine-chelated zinc, isoleucine-chelated zinc and combinations thereof.

13. A combination, comprising:
an amino acid-chelated zinc; and
a dendritic cell medium.

14. The combination of claim 13, wherein the amino acid-chelated zinc has a molar ratio of zinc and amino acid ranging from 1:1 to 1:10.

15. The combination of claim 13, wherein the amino acid-chelated zinc is selected from the group consisting of glycine-chelated zinc, alanine-chelated zinc, valine-chelated zinc, leucine-chelated zinc, isoleucine-chelated zinc and combinations thereof.

16. The combination of claim 13, further comprising a dendritic cell.

17. The combination of claim 13, wherein the amino acid-chelated zinc has an amount of zinc ranging from 1 mg to 25 mg per mL of the dendritic cell medium.

18. The combination of claim 14, wherein the amino acid-chelated zinc has an amount of zinc ranging from 1 mg to 25 mg per mL of the dendritic cell medium.

19. The combination of claim 15, wherein the amino acid-chelated zinc has an amount of zinc ranging from 1 mg to 25 mg per mL of the dendritic cell medium.

20. The combination of claim 16, wherein the amino acid-chelated zinc has an amount of zinc ranging from 1 mg to 25 mg per mL of the dendritic cell medium.
